(19) United States
(12) Patent Application Publication
(10) Pub. No.: US 2017/0129863 A1
(43) Pub. Date: May 11, 2017

(54) IMINOSYDNONE DERIVATIVES FOR CONJUGATION AND RELEASE OF COMPOUNDS OF INTEREST

(71) Applicants: UNIVERSITE DE STRASBOURG, STRASBOURG (FR); CENTRE NATIONAL DE LA RECHERCHE SCIENTIFIQUE, PARIS (FR); COMMISSARIAT A L'ENERGIE ATOMIQUE ET AUX ENERGIES ALTERNATIVES, PARIS (FR)

(72) Inventors: FREDERIC TARAN, GIF SUR YVETTE (FR); ALAIN WAGNER, STRASBOURG (FR); SERGII KOLODYCH, STRASBOURG (FR); OLEKSANDR KONIEV, STRASBOURG (FR)

(21) Appl. No.: 15/319,387

(22) PCT Filed: Jun. 18, 2015
(86) PCT No.: PCT/EP2015/063755

§ 371 Date: Dec. 16, 2016

(30) Foreign Application Priority Data
Jun. 18, 2014 (EP) 14305933.5

(51) Int. Cl. C07D 271/04; C07D 413/12

(52) U.S. Cl. CPC C07D 271/04; C07D 413/12

(57) ABSTRACT

The present invention relates to the use of iminosydnone compounds in processes for the preparation of conjugates of two compounds of interest. The invention further relates to the use of said iminosydnone compounds in a process for releasing a compound of interest. The invention finally relates to novel iminosydnone compounds.

### INTRODUCTION

The present invention relates to the use of iminosydnone compounds in processes for the preparation of conjugates of two compounds of interest. The invention further relates to the use of said iminosydnone compounds in a process for releasing a third compound of interest. The invention finally relates to said new iminosydnone compounds.

### BACKGROUND OF THE INVENTION

The present invention relates to the domain of click chemistry (bioorthogonal reaction) and of bioconjugation of compounds of interest. Five main approaches are currently available for metal-free coupling two different compounds of interest ( and ▪). These approaches are presented on the scheme below. Among these approaches only Tetrazine/Cyclooctene reaction (approach 2) has a modification that allows simultaneous coupling of two entities and release of the third compound (Angew. Chem. Int. Ed. 2013, 52, 14112-14116).

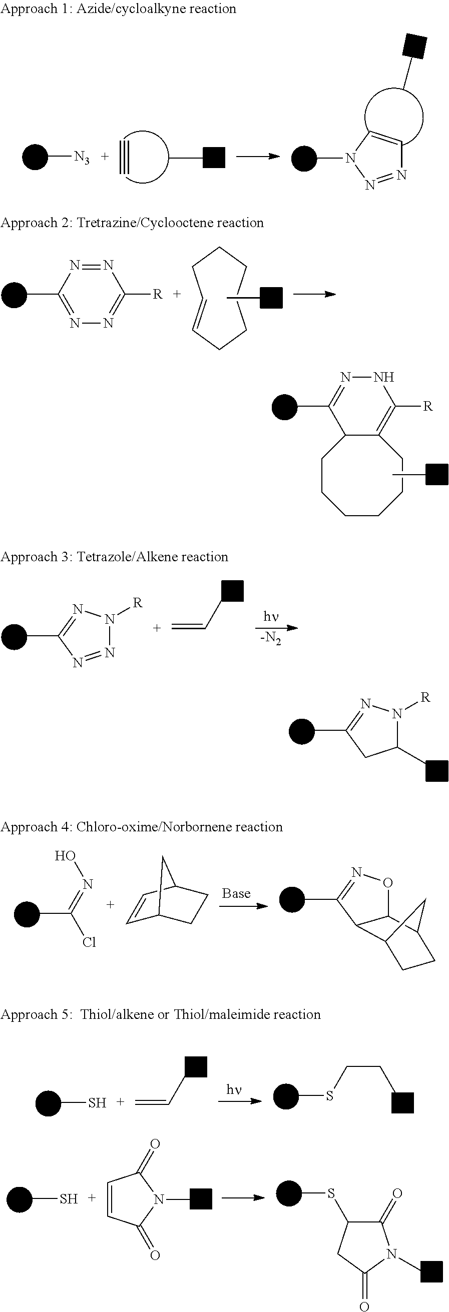

The inventors of the present application recently discovered that reaction of a sydnone and an alkyne could be efficiently used to perform efficient bioorthogonal reactions (Kolodych et al. Angew. Chem. Int. Ed. 2013, 52, 12056-12060).

Bioconjugation reactions and cleavage reactions, for instance bioconjugation and cleavage reactions involving proteins, are often performed in highly dilute conditions, for instance because of the low available amount of the starting biomaterial, or because the solubility of said starting biomaterial is low in the coupling and or cleavage conditions. Furthermore, a high level of bioorthogonality of these reactions is mandatory for applications in the fields of chemical biology and drug release which deal with in vivo experiments.

Consequently, it is important in the domain of bioorthogonal reactions to use efficient coupling and cleavable reagents allowing high yields and fast coupling and cleavage kinetics even in the presence of the plethora of chemical functionalities present in biological media.

Such reactions are potentially of high interest in several areas; one of them is antibody-drug-conjugate (ADC) cleavage wherein an antibody, such as an anti-tumor antibody, and a drug, such as an anticancer drug, can be selectively released with a specific probe preferably administered in a second step.

The Applicant of the present invention evidenced that the use of iminosydnone derivatives affords a unique process allowing both the conjugation and the release of compounds of interest with high efficiency, kinetics and bioorthogonality. These iminosydnone compounds can be advantageously used for the conjugation and the release of compounds of interest.

### SUMMARY OF THE INVENTION

A first object of the invention is a process for the preparation of a functionalized compound of interest C1 of formula (II):

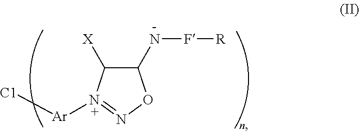

comprising the step of contacting a compound of interest C1 bearing a reactive group with an iminosydnone of formula (I):

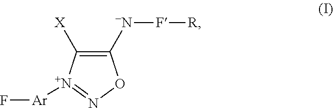

wherein F is a functional group selected among specific groups which allow bonding to the reactive group of the compound of interest C1.

Another object of the invention is a functionalized compound of interest C1 of formula (II):

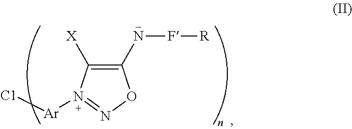

Another object of the invention is a conjugate of formula (IV):

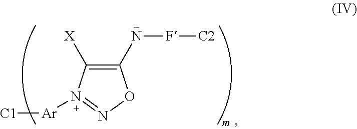

wherein C1 and C2 are compounds of interest.

Another object of the invention is a process for the preparation of a conjugate of formula (IV):

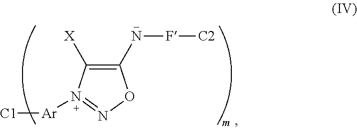

comprising the step of contacting a compound of formula (II) with a compound of interest C2 bearing a reactive group which is able to react with R.

Another object of the invention is a process for the preparation of a conjugate of formula (V):

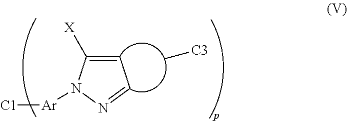

wherein C3 is a third compound of interest, comprising a step of contacting a compound of formula (VI):

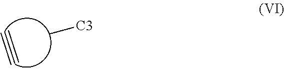

with the compound of formula (II) or with the conjugate of formula (IV).

Another object of the invention is a conjugate of formula (V):

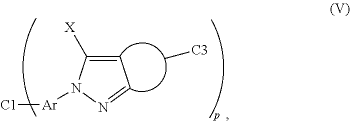

A further object of the invention is an iminosydnone of formula (IT

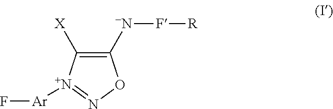

### BRIEF DESCRIPTION OF THE FIGURES

FIG. 1: HPLC monitoring of the reaction between iminosydnone Im 5 and the cyclic alkyne TMTH.

FIG. 2: HPLC monitoring (b) and linear regression curve (c) for the reaction of Im2 with TMTH (a).

FIG. 3: Linear regression curves for the reactions of iminosydnones Im6 and Im7 with TMTH.

### DETAILED DESCRIPTION OF THE INVENTION

A first object of the present invention is a process for the preparation of a functionalized compound of interest C1 of formula (II)

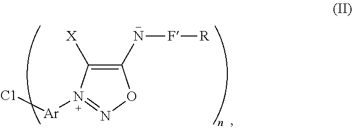

wherein n is an integer and preferably ranges from 1 to 100, comprising the step of contacting a compound of interest C1 selected from an antibody, a protein, a drug, a fluorophore, a group of atoms comprising at least one radioactive atom, a DNA fragment, a nanoparticle and a polymer, with an iminosydnone of formula (I):

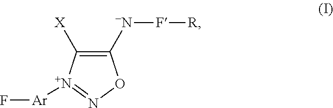

wherein
C1 bears a reactive group which is able to react with F,
X is selected from the group consisting of a hydrogen atom, a halogen atom, an aryl diazo group, an alkyl group, an aryl group, an alkenyl group, an alkynyl group, an alkoxy group, a thioether group and an amino group,
F is a functional group selected from the group consisting of:
a carboxylic acid COOH group,
a thiol SH group,
a maleimide

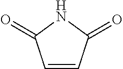

group,
an activated ester,
a halogen atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups,
a hydroxylamine (—ONH₂) group,
a hydrazine (—NH—NH₂) group,
an azido (—N₃) group,
a diazonium (—N₂⁺) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′″ group, wherein R′″ is an alkyl group, and
an alkyl group substituted by at least one of said groups,
   F′ is a carbonyl group (C═O), a sulfonyl group (SO₂) or a phosphoryl group (P═O),
   R is selected from
   an optionally substituted aryl group, an optionally substituted alkyl, alkenyl or alkynyl group, an optionally substituted alkoxy or aralkyloxy group, an optionally substituted thioether group, an optionally substituted amino group, wherein the alkyl, alkenyl and/or alkynyl groups may be interrupted by at least one heteroatom selected from nitrogen, oxygen and sulphur atoms and wherein said substituents are one or more groups selected from:
a carboxylic acid COOH group,
a thiol SH group,

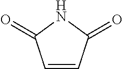

a maleimide group,
an activated ester,
a halogen atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups,
a hydroxylamine (—ONH₂) group,
a hydrazine (—NH—NH₂) group,
an azido (—N₃) group,
a diazonium (—N₂⁺) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′″ group, wherein R′″ is an alkyl group, or
   a linker bearing at least one of the above substituents, and
   Ar is an optionally substituted aromatic group.

In an embodiment, n is from 1 to 50, preferably from 1 to 30. In a specific embodiment, n is 1.

In the above definitions of F and R, the counterion can be any ion appropriate for compensating the charge of the diazonium group, and may be easily chosen by anyone of ordinary skill in the art. For instance, the couterion may be selected from the group consisting of halogenates, BF₄, NO₃⁻, HSO₄⁻, PF₆⁻CH₃COO⁻, N(SO₂CF₃)₂⁻, CF₃SO₃⁻, CH₃SO₃⁻, CF₃COO⁻, (CH₃O)(H)PO₂⁻ and N(CN)₂⁻.

In the context of this description, a halogen atom is a chlorine, iodine, bromine or fluorine atom. Preferably, a halogen atom is a bromine or a chlorine atom, in particular a bromine atom. An alkyl group is a linear saturated hydrocarbon group comprising from 1 to 20 carbon atoms, a branched saturated hydrocarbon group comprising from 3 to 20 carbon atoms or a cyclic saturated hydrocarbon group comprising from 4 to 20 carbon atoms. Preferably, the alkyl group according to the invention comprises from 1 to 10, in particular from 1 to 6, carbon atoms. Examples of alkyl groups comprise methyl, ethyl, propyl, isopropyl, butyl, tertbutyl, isobutyl, n-pentyl, n-hexyl and cyclohexyl groups.

An alkenyl group (or an alkene) is a linear hydrocarbon group comprising from 2 to 20 carbon atoms, a branched hydrocarbon group comprising from 4 to 20 carbon atoms, or a cyclic hydrocarbon group comprising from 4 to 20 carbon atoms and comprising at least one C═C double bond. Preferably, the alkenyl group according to the invention comprises from 2 to 10, in particular from 2 to 6, carbon atoms. Examples of alkenyl groups comprise ethylenyl, propylenyl and cyclohexenyl groups.

An alkynyl group (or an alkyne) is a linear hydrocarbon group comprising from 2 to 20 carbon atoms, a branched hydrocarbon group comprising from 4 to 20 carbon atoms or a cyclic hydrocarbon group comprising from 4 to 20 carbon atoms and comprising at least one C≡C triple bond. Preferably, the alkynyl group according to the invention comprises from 2 to 10, in particular from 2 to 9, carbon atoms. Examples of alkynyl groups comprise ethynyl, propynyl, octynyl, cyclooctynyl and cyclononynyl groups.

In specific embodiments of the present invention, the alkyl, alkenyl and/or alkynyl groups may be interrupted by at least one heteroatom, preferably independently selected from nitrogen, oxygen and sulphur atoms.

An alkoxy group is an alkyl group, bonded to the rest of the molecule through an oxygen atom.

An aralkyloxy group is an aralkyl group, such as a benzyl group, bonded to the rest of the molecule through an oxygen atom.

A thioether group is an alkyl group, bonded to the rest of the molecule through a sulfur atom.

An aryl diazo group is a N₂ group bonded to an aromatic group. A carboxyl group is a COOH group. A nitro group is a NO₂ group.

An amino group is a NR₁R₂ group, wherein R₁ and R₂ are independently selected from the group consisting of hydrogen atoms, aromatic groups (such as a phenyl or tolyl group) and alkyl groups. In an embodiment, at least one of R₁ and R₂ is an alkyl group. Such an amino group is an alkylamino group. In an embodiment, R₁ and R₂ are both alkyl groups. Such an amino group is a dialkylamino group.

An “activated ester” is an ester with a good leaving group, such as a N-hydroxysuccinimide ester, a N-hydrophthalimide ester, a perfluorinated ester or an acylurea. An example of perfluorinated ester is the following:

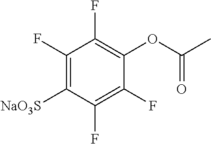

According to the present invention, the optionally substituted aromatic group Ar is selected from the group consisting of:
a phenyl group;
a heteroaromatic group, such as pyridine, thiophene, imidazole, thiazole, pyrazole, pyrole or furane; and
a polyaromatic group, such as anthracene or phenanthrene.

The phenyl, heteroaromatic and/or polyaromatic group may be substituted with at least one substituent (in addition to the F group when the aromatic is substituted with a F group), preferably selected from the group consisting of a halogen atom, an alkyl group, an alkoxy group, a carboxyl COOH group, a COOR₃ group, wherein R₃ is an alkyl group, and a nitro NO₂ group.

In an embodiment, the optionally substituted aromatic group is an optionally substituted phenyl group, preferably a non-substituted phenyl group.

The linker optionally comprised in R may be any hydrocarbon chain comprising C and H atoms and optionally O and/or N and/or S atoms, such as a —(CH₂)q(CH₂O)r-COO—(CH₂)q′(CH₂O)r′ group, a —(CH₂)q(CH₂O)r-NH—C(═O)—(CH₂)q′(CH₂O)r′ group, a —(CH₂)q(CH₂O)r-CONH—(CH₂)q′(CH₂O)r′ group, wherein q and r are integers independently ranging from 0 to 10, or a triazolyl group, for instance.

In an embodiment, X is a halogen atom.

When Ar is an optionally substituted phenyl group, the functional group F is in meta or para position of the phenyl group, preferably in para position.

When Ar is an optionally substituted heteroaromatic or polyaromatic group, the functional group F can be in any position of the aromatic group.

In a preferred embodiment, the functional group F is selected from the group consisting of:
a carboxylic acid COOH group,
a thiol SH group,
a maleimide

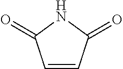

group,
an activated ester,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
an isocyanate (—N═C═O) or isothiocyanate (—N═C═S) group,
a chlorosulfonyl (—SO₂Cl) group,
a —C≡C—C≡N group, and
an alkyl group substituted by at least one of the groups above.

In a highly preferred embodiment, the functional group F is selected from the group consisting of a carboxylic acid COOH group, an activated ester, such as a N-hydroxysuccinimide, a N-hydrophthalimide ester, a perfluorinated ester or an acylurea, and an alkyl group substituted by at least one of these groups.

In a preferred embodiment, F′ is a C═O group and R is an amino group. Preferably, the R amino group is bonded through the nitrogen atom to the C═O F′ group, thus forming a urea moiety.

In another preferred embodiment, F′ is a C═O group and R is an alkoxy group such as an tert-butoxy group. Preferably, the R alkoxy group is bonded through the oxygen atom to the C═O F′ group, thus forming a carbamate moiety.

In a preferred embodiment, F′ is a C═O group, R is an amino or alkoxy group forming a urea or carbamate moiety respectively as disclosed above, and X is a halogen atom, preferably selected from bromine and chlorine atoms.

Examples of iminosydnones of formula (I) that may be used in the above process are selected from the group consisting of:
(2-(4-hydroxyphenyl)-1,2,3-oxadiazol-2-ium-5-yl)(p-tolylcarbamoyl)amide;
(2-(4-carboxyphenyl)-1,2,3-oxadiazol-2-ium-5-yl)(p-tolylcarbamoyl)amide; and
((4-(ethoxycarbonyl)phenyl)carbamoyl)(2-(4-hydroxyphenyl)-1,2,3-oxadiazol-2-ium-5-yl)amide.

In an embodiment, n is from 1 to 50, preferably from 1 to 30. In a specific embodiment, n is 1.

The contacting step of the processes according to the invention may be performed by any technique known in the art, for instance by dissolution and/or dispersion of both compounds in a solvent. The contacting may be performed under stirring, for instance mechanical or magnetic stirring. The contacting may be performed at room temperature, ie at a temperature comprised between 18 and 25° C., or at low temperature, ie at a temperature inferior to 18° C., or even at high temperature, ie at a temperature higher than 25° C., for instance comprised between 25 and 150° C.

The process of preparation of a functionalized compound according to the invention may comprise a preliminary step of covalent bonding of a reactive group to C1, in the case where C1 does not bear a reactive group able to react with the functional group F of the iminosydnone compound. However, in a preferred embodiment, C1 intrinsically bears such a reactive group. In an embodiment, the reactive group is selected from the group consisting of:
a carboxylic acid COOH group,
a thiol SH group,
a maleimide

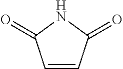

group,
an activated ester
a halogen atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
a diazonium (—N₂⁺) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′″ group, wherein R′″ is an alkyl group.

In a preferred embodiment, the reactive group is selected from the group consisting of:
a carboxylic acid COOH group,
a thiol SH group,
a maleimide

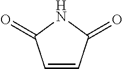

group,
an activated ester,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
an isocyanate (—N═C═O) or isothiocyanate (—N═C═S) group,
a chlorosulfonyl (—SO₂Cl) group, and
a —C≡C—C≡N group.

In a highly preferred embodiment, the reactive group is an amino group or a thiol group.

The racemic forms, tautomers, enantiomers, diastereoisomers, epimers, solvates and salts of the compounds used in the processes according to the invention are also part of the scope of the invention.

As examples of salts may be cited acid addition salts, such as hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically acceptable inorganic or organic acid addition salts include the pharmaceutically acceptable salts listed in J. Pharm. Sci. 1977, 66, 2, and in Handbook of Pharmaceutical Salts: Properties, Selection, and Use edited by P. Heinrich Stahl and Camille G. Wermuth 2002.

Another object of the invention is a functionalized compound of interest C1 of formula (II):

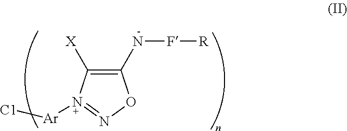

wherein C1, Ar, X, F′, n and R are as defined above and C1 and Ar are covalently linked by a functional group. This functional group results from the reaction between the reactive group of C1 and the functional group F, such as an ester or amide function.

Another object of the invention is a conjugate of formula (IV):

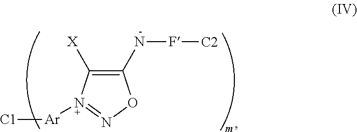

wherein X, Ar, F′ and C1 are as described above, m is an integer which ranges from 1 to 100, C2 is a compound of interest selected from an antibody, a protein, a drug, a fluorophore, a group of atoms comprising at least one radioactive atom, a DNA fragment, a nanoparticle and a polymer C1 and Ar are covalently linked by a functional group (resulting from the reaction between the reactive group of C1 and the functional group F) and C2 and F′ are covalently linked by a functional group. This functional group results from the reaction between the reactive group of C2 and R and may be for instance as an ester or amide function. In an embodiment, m is from 1 to 50, preferably from 1 to 30. In a specific embodiment, m is 1.

Another object of the invention is a process for the preparation of a conjugate of formula (IV) as described above, comprising the step of contacting a compound of formula (II) with a compound of interest C2 bearing a reactive group which is able to react with R. It is understood that, in this embodiment of the invention, R is itself a reactive group.

Specific examples of R for use in this embodiment are:
a carboxylic acid COOH group,
a thiol SH group,
a maleimide

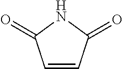

group,
an activated ester
an amino (—NRR′) group, wherein R and R′ are independently hydrogen atoms, alkyl, alkene, alkyne or aryl groups as defined above, and
a —C≡C—C≡N group.

The process of preparation of the above conjugate may comprise a preliminary step of covalent bonding of a reactive group to C2, in the case where C2 does not bear a reactive group able to react with the reactive group R of the iminosydnone compound. However, in a preferred embodiment, C2 intrinsically bears such a reactive group.

In an embodiment, this reactive group of C2 is selected from the group consisting of:
a carboxylic acid COOH group,
a thiol SH group,
a maleimide

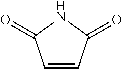

group,
an activated ester
a halogen atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
a diazonium (—N²) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′″ group, wherein R′″ is an alkyl group.

In a preferred embodiment, the reactive group is selected from the group consisting of:
a carboxylic acid COOH group,
a thiol SH group,
a maleimide

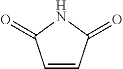

group,
an activated ester,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
an isocyanate (—N═C═O) or isothiocyanate (—N═C═S) group,
a chlorosulfonyl (—SO₂Cl) group, and
a —C≡C—C≡N group.

In a highly preferred embodiment, the reactive group is an amino group or a thiol group. Another object of the invention is a process for the preparation of a conjugate of formula (V):

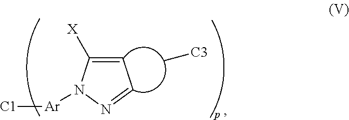

wherein p is an integer ranging from 1 to 100, C1, Ar and X are as defined above, C1 is a first compound of interest, and C3 is a third compound of interest selected from an antibody, a protein, a drug, a fluorophore, a group of atoms comprising at least one radioactive atom, a DNA fragment, a nanoparticle and a polymer, comprising a step of contacting a compound of formula (VI):

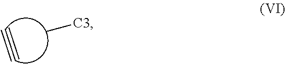

with a compound of formula (II) or with a conjugate of formula (IV) according to the invention. In an embodiment, p is from 1 to 50, preferably from 1 to 30. In a specific embodiment, p is 1. In a preferred embodiment where the compound of formula (VI) is reacted with a conjugate of formula (IV), this process simultaneously allows the release of a derivative of C2 of formula (VII):

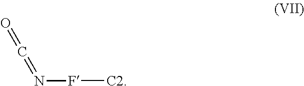

The process for the preparation of a conjugate of formula (V) may comprise a preliminary step of covalent bonding of a strained alkyne moiety to the compound of interest C3.

By “strained alkyne”, it is meant a cyclic alkyne, in particular cyclooctynes such as bicyclo-[6.1.0]-nonyne (BCN) or cycloheptynes such as tetramethylthiacycloheptyne (TMTH). In the case of BCN, for instance, the compound of interest C3 may be bonded to BCN by reacting a C3-O—CO—Cl compound with the hydroxy group of BCN, so as to form a carbamate bond. In the case of TMTH, the latter may be alkylated on its sulphur atom by reaction with a C3-CH2-Br compound.

Another object of the invention is a conjugate of formula (V):

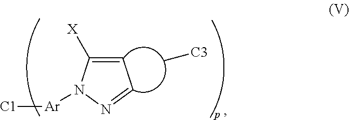

wherein C1, Ar, X and C3 are as defined above.

Another object of the invention is a process for releasing a derivative of C2 of formula (VII):

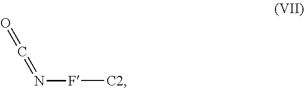

comprising a step of contacting a conjugate of formula (IV):

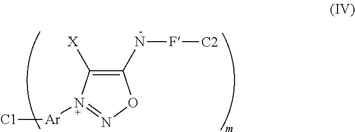

as described above with a compound comprising a strained alkyne moiety, preferably a cyclic alkyne moiety, in particular a cyclooctyne moiety, for instance BCN (bicyclononyne). More preferably, the conjugate of formula (IV) is reacted with a compound of formula (VI) as described above.

Another object of the invention is an iminosydnone of formula (I′):

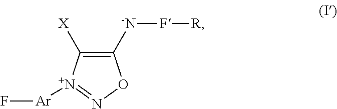

wherein
X is a halogen atom,
F, F′, R and Ar are as described above.

The iminosydnone compounds of formulas (I) and (I′) can be used advantageously in order to couple both compounds of interest C1 and C3 or C1 and C2 and/or release the compound of interest C2, as explained above.

In an embodiment, C1 is a nanoparticle or an antibody, C2 is a drug and C3 is a fluorophore or a group of atoms comprising at least one radioactive atom. In this embodiment, the iminosydnone of the invention affords the release of the drug, which may then enter its target cells, and the labeling of the nanoparticle or the antibody. In a particular embodiment, C1 is an antibody and this embodiment leads to the release of a drug from a therapeutic antibody and at the same time the labeling of the antibody, therefore allowing theranostic applications. In an especially preferred embodiment of this invention, the drug is a chemotherapeutic drug and the labeling of the antibody allows imaging the tumor.

The iminosydnones of formulas (I) and (I′) react very efficiently with strained alkynes, such as cyclic alkynes, in particular cyclooctynes. The coupling reaction implies the formation of a cycloadduct formed by a [3+2] cyclization turning to a stable pyrazole said cycloadduct by retro-Diels Alder reaction, triggering the release of a R—F′—NCO molecule, which in aqueous medium, for instance in water, may spontaneously evolve to R—F′—NH₂ through hydrolysis and/or decarboxylation reactions. The reaction with a compound according to the invention with a cycloalkyne is represented as example on the scheme below.

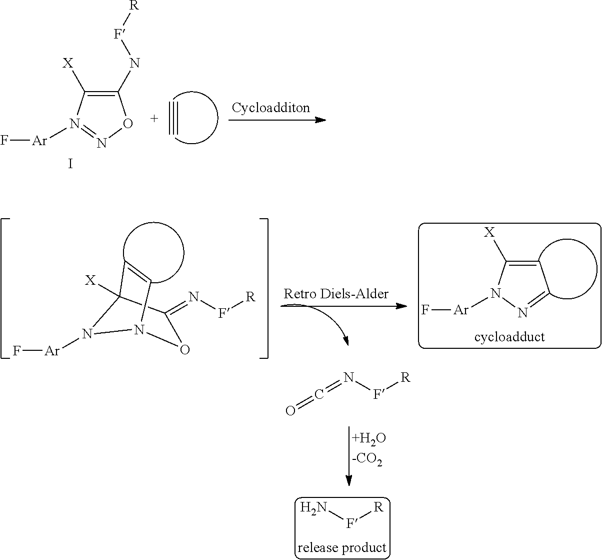

The present compounds allow the efficiency of the reaction to be maintained in biological media, such as culture media, cell lysates or plasma.

These iminosydnones may be synthesized by any appropriate method known by one of ordinary skill in the art.

For instance, the scheme below presents different synthesis routes for iminosydnones of formulas (I) and (I′). The Ar groups substituting the nitrogen atom of the iminosydnones may be substituted with a F group. In this scheme, the term NBS designates N-bromosuccinimide.

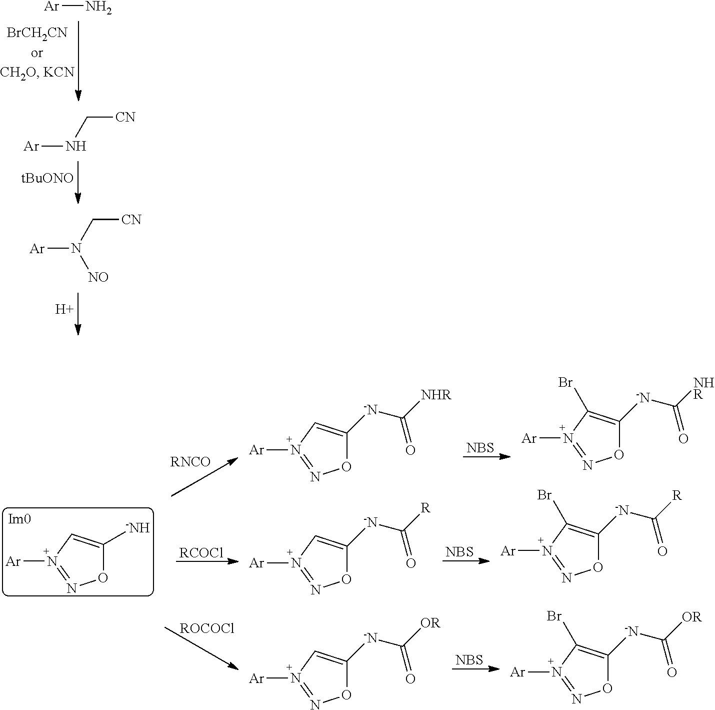

The following examples are provided as illustrative, and not limitative, of the present invention.

### Examples

### Example 1: Synthesis of Sydnones

In the following examples, the provided yields are molar yields unless specified differently.

### Im0-1 (intermediate): 5-amino-3-(4-hydroxyphenyl)-1,2,3-oxadiazol-3-ium chloride

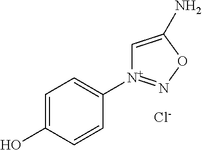 [SMILES: Nc1c[n+](-c2ccc(O)cc2)no1 ; [Cl-]]

To a solution of 2-((4-hydroxyphenyl)amino)acetonitrile (2.56 g, 12 mmol) in tetrahydrofuran THF (60 mL) was added amyl nitrite (1.85 eq, 22.2 mmol). The mixture was stirred for 16 h at room temperature (r.t.) and then HCl (5 mL, 4M solution in dioxane) was added. The resulting mixture was stirred for 24 h at r.t. The precipitate was collected by filtration and washed with Et₂O and dried to yield pure product as a yellow solid (850 mg, 4.8 mmol, 40%).

¹H NMR (400 MHz, DMSO-d₆, δ ppm): 9.71 (s, 2H), 8.48 (s, 1H), 7.88 (d, J=9.1 Hz, 2H), 7.09 (d, J=9.1 Hz, 2H). ¹³C NMR (100 MHz, DMSO-d₆, δ ppm): 169.1, 162.1, 124.1, 124.0, 116.6, 101.0.

### Im0-2 (intermediate): 5-amino-3-(4-carboxyphenyl)-1,2,3-oxadiazol-3-ium chloride

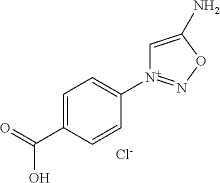 [SMILES: Nc1c[n+](-c2ccc(C(=O)O)cc2)no1 ; [Cl-]]

To a solution of 4-((cyanomethyl)amino)benzoic acid (1.13 g, 6.42 mmol) in THF (60 mL) was added amyl nitrite (1.85 eq, 11.9 mmol). The mixture was stirred for 16 h at r.t. and then HCl (5 mL, 4M solution in dioxane) was added. The resulting mixture was stirred for 24 h at r.t. The precipitate was collected by filtration and washed with Et₂O and dried to yield pure product as a white solid (161 mg, 0.67 mmol, 10%).

¹H NMR (400 MHz, DMSO-d₆, δ ppm): 9.97 (br. s., 2H), 8.73 (s, 1H), 8.27 (d, J=8.6 Hz, 2H), 8.18 (d, J=8.6 Hz, 2H). ¹³C NMR (100 MHz, DMSO-d₆, δ ppm): 169.4, 165.7, 135.5, 135.1, 131.0, 123.1, 102.7.

### Im 6 (comparative): (2-(4-hydroxyphenyl)-1,2,3-oxadiazol-2-ium-5-yl)(p-tolylcarbamoyl)amide

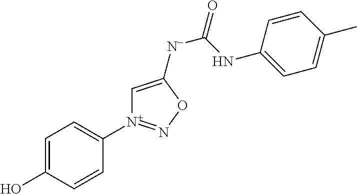 [SMILES: Cc1ccc(NC(=O)[N-]c2c[n+](-c3ccc(O)cc3)no2)cc1]

To a solution of p-tolyl isocyanate (47 mg, 0.351 mmol) and iminosydnone Im0-1 S (75 mg, 0.351 mmol) in THF (5 mL) was added a solution of NaHCO₃ (1 eq., 30 mg, 0.35 mmol) in H₂O (1 mL). The resulting solution was stirred at room temperature for 14 hours. The organic layer was separated, dried over MgSO₄ and evaporated. The residue was purified by semi-preparative HPLC (MeCN/H₂O gradient) to afford Im6 as a white solid (18.3 mg, 0.059 mmol, 17%).

¹H NMR (400 MHz, DMSO-d₆, δ ppm): 9.29 (br. s., 1H), 8.38 (s, 1H), 7.89 (d, J=9.0 Hz, 2H), 7.54 (d, J=8.2 Hz, 2H), 7.07-6.93 (m, J=6.5, 8.5 Hz, 4H), 2.22 (s, 3H).

¹³C NMR (100 MHz, DMSO-d₆, δ ppm): 172.2, 161.1, 158.8, 138.5, 129.9, 128.8, 125.3, 123.7, 117.9, 116.4, 102.1, 20.4.

### Im 8 (comparative): ((4-(ethoxycarbonyl)phenyl)carbamoyl)(2-(4-hydroxyphenyl)-1,2,3-oxadiazol-2-ium-5-yl)amide

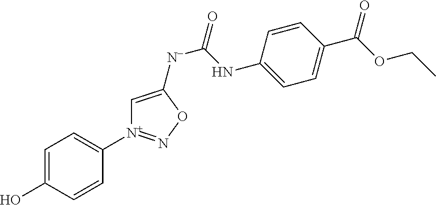 [SMILES: CCOC(=O)c1ccc(NC(=O)[N-]c2c[n+](-c3ccc(O)cc3)no2)cc1]

To a solution of ethyl 4-isocyanatobenzoate (67 mg, 0.351 mmol) and iminosydnone Im0-1 (75 mg, 0.351 mmol) in THF (5 mL) was added a solution of NaHCO₃ (1 eq., 30 mg, 0.35 mmol) in H₂O (1 mL). The resulting solution was stirred at room temperature for 14 hours. The organic layer was separated, dried over MgSO₄ and evaporated. The residue was purified by semi-preparative HPLC (MeCN/H₂O gradient) to afford Im 8 as a yellow solid (16.1 mg, 0.044 mmol, 13%).

¹H NMR (400 MHz, DMSO-d₆, δ ppm): 10.60 (br. s., 1H), 9.82 (s, 1H), 8.49 (s, 1H), 7.91 (d, J=9.0 Hz, 2H), 7.84 (d, J=8.8 Hz, 2H), 7.77 (d, J=8.8 Hz, 2H), 7.02 (d, J=9.0 Hz, 2H), 4.27 (q, J=7.0 Hz, 2H), 1.30 (t, J=7.0 Hz, 3H).

¹³C NMR (100 MHz, DMSO-d₆, δ ppm): 172.4, 165.5, 161.1, 158.6, 145.6, 130.0, 125.2, 123.7, 122.1, 117.0, 116.3, 102.6, 60.0, 14.2.

MS (ESI) m/z: 369.2 [M+H]⁺.

### Im 7: (2-(4-carboxyphenyl)-1,2,3-oxadiazol-2-ium-5-yl)(p-tolylcarbamoyl)amide

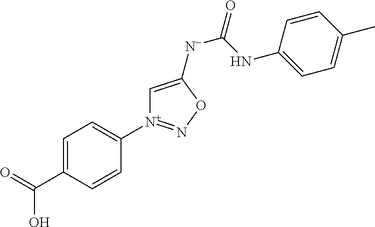 [SMILES: Cc1ccc(NC(=O)[N-]c2c[n+](-c3ccc(C(=O)O)cc3)no2)cc1]

To a solution of p-tolyl isocyanate (15 mg, 0.113 mmol) and iminosydnone Im0-2 (27 mg, 0.113 mmol) in THF (5 mL) was added a solution of NaHCO₃ (1 eq., 10 mg, 0.12 mmol) in H₂O (1 mL). The resulting solution was stirred at room temperature for 14 hours. The organic layer was separated, dried over MgSO₄ and evaporated. The residue was purified by semi-preparative HPLC (MeCN/H₂O gradient) to afford Im 7 as a yellow solid (8.6 mg, 0.025 mmol, 22%).

¹H NMR (400 MHz, DMSO-d₆, 6 ppm): 9.41 (br. s., 1H), 8.69 (s, 1H), 8.21 (s, 4H), 7.53 (d, J=8.1 Hz, 2H), 7.04 (d, J=8.1 Hz, 2H), 2.22 (s, 3H).

¹³C NMR (100 MHz, DMSO-d₆, δ ppm): 171.8, 165.9, 157.9, 138.1, 136.5, 134.5, 131.0, 130.2, 128.8, 122.6, 118.1, 103.5, 20.3.

MS (ESI) m/z: 339.1 [M+H]⁺.

### Im13: (3-(4-carboxyphenyl)-1,2,3-oxadiazol-3-ium-5-yl)((2-(3-(2,5-dioxo-2,5-dihydro-1H-pyrrol-1-yl)propanamido)ethyl)carbamoyl)amide

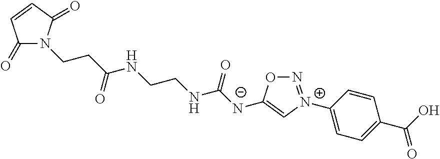 [SMILES: O=C(CCN1C(=O)C=CC1=O)NCCNC(=O)[N-]c1c[n+](-c2ccc(C(=O)O)cc2)no1]

The following synthesis process was conducted to prepare the above compound, referred to as K:

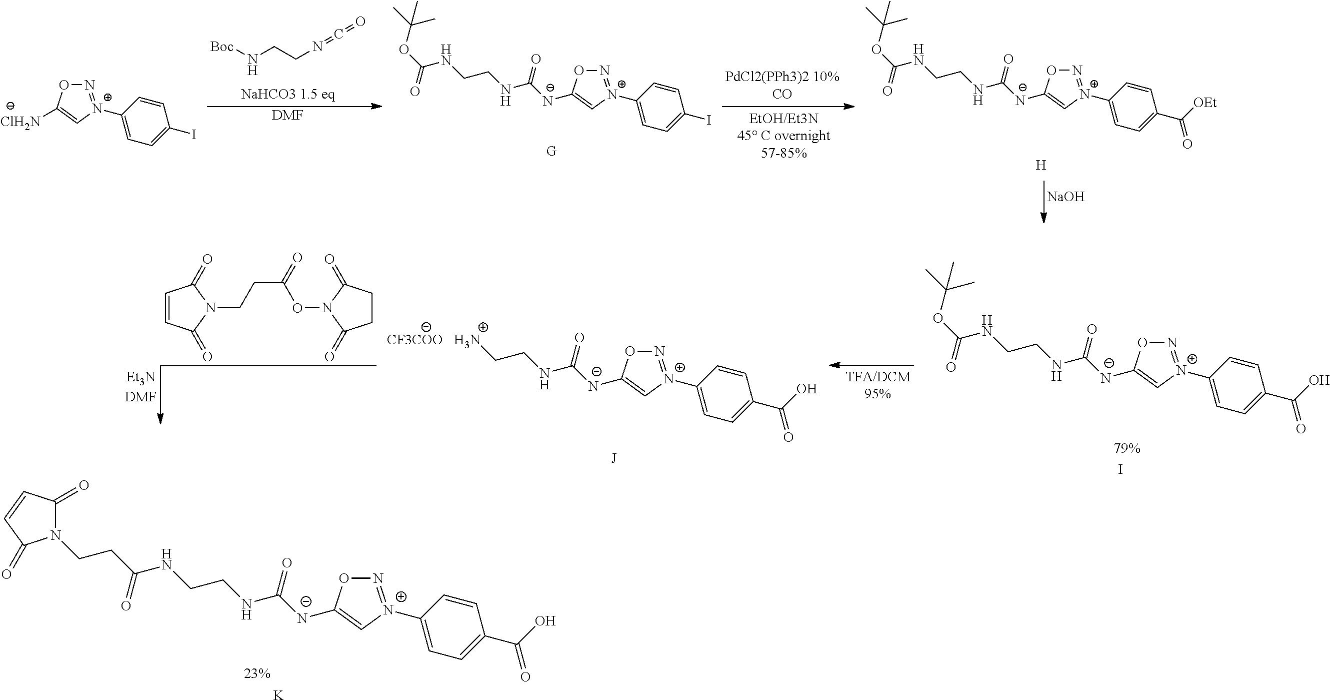

### Step (a): synthesis of G-((2-((tert-butoxycarbonyl)amino)ethyl)carbamoyl)(3-(4-iodophenyl)-1,2,3-oxadiazol-3-ium-5-yl)amide

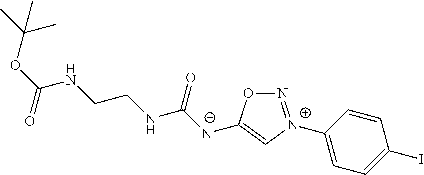 [SMILES: CC(C)(C)OC(=O)NCCNC(=O)[N-]c1c[n+](-c2ccc(I)cc2)no1]

To a suspension of 5-amino-3-(4-iodophenyl)-1,2,3-oxadiazol-3-ium chloride and NaHCO₃ in dry DMF (1 mL) was added the tert-butyl (2-isocyanatoethyl)carbamate. The mixture stirred at room temperature overnight. The mixture was quenched with NH4Cl (15 mL) then extracted with AcOEt. The combined organic layers were dried (MgSO4) and concentrated under reduced pressure. The crude material was though flash chromatography.

¹H NMR (CDCl₃, 400 MHz, ppm): δ=8.14 (s, 1H), 7.98 (d, J=8.4 Hz, 2H), 7.52 (d, J=8.4 Hz, 2H), 5.77 (bs, 1H), 5.07 (bs, 1H), 3.40-3.36 (m, 2H), 3.30-3.26 (m, 2H), 1.42 (s, 9H);

¹³C NMR (CDCl₃, 101 MHz, ppm): δ=172.6, 161.9, 156.1, 139.6 (2C), 133.5, 122.7 (2C), 101.7, 99.2, 79.2, 41.0, 40.0, 28.3 (3C);

IR (v, cm⁻¹): 3283, 2976, 1691, 1599, 1632, 1515, 1430, 1365, 1274, 1220, 1007, 959;

MS (ESI) m/z: [M+H]⁺=474.2.

### Step (b): synthesis of H-((2-((tert-butoxycarbonyl)amino)ethyl)carbamoyl)(3-(4-(ethoxycarbonyl)phenyl)-1,2,3-oxadiazol-3-ium-5-yl)amide

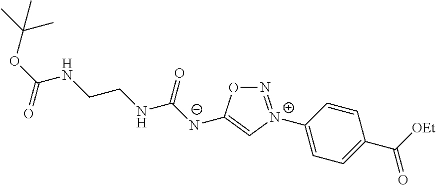 [SMILES: CCOC(=O)c1ccc(-[n+]2cc([N-]C(=O)NCCNC(=O)OC(C)(C)C)on2)cc1]

((2-((tert-butoxycarbonyl)amino)ethyl)carbamoyl)(3-(4-iodophenyl)-1,2,3-oxadiazol-3-ium-5-yl)amide (45 mg, 0.095 mmol, 1 equiv.) was dissolved in a mixture of absolute EtOH (1 mL) and triethylamine (1 mL). [PdCl₂(PPh₃)₂] (6.3 mg, 0.009 mmol, 10 mol %) was added and the reaction mixture was kept under a CO atmosphere for 15 h and heated at 45° C. The resulting suspension was cooled to room temperature and filtered through Celite eluting with ethyl acetate, and the inorganic salts were removed. The filtrate was concentrated and purification of the residue by silica gel column chromatography gave the desired product.

¹H NMR (CDCl₃, 400 MHz, ppm): 8=8.31 (d, J=8.6 Hz, 2H), 8.20 (s, 1H), 7.88 (d, J=8.6 Hz, 2H), 5.76 (bs, 1H), 5.03 (bs, 1H), 4.45 (q, J=7.1 Hz, 2H), 3.40-3.36 (m, 2H), 3.30-3.25 (m, 2H), 1.45-1.41 (m, 12H);

¹³C NMR (CDCl₃, 101 MHz, ppm): 8=172.5, 164.4, 161.7, 156.1, 136.7, 134.5, 131.5 (2C), 121.4 (2C), 102.1, 79.1, 61.9, 41.0, 40.4, 28.3 (2C), 14.2;

IR (v, cm⁻¹): 3288, 1978, 1713, 1635, 1604, 1512, 1441, 1366, 1274, 1173, 1108, 958, 770;

MS (ESI) m/z: [M+H]⁺=420.1;

Mp: 139-141° C.

### Step (c): synthesis of I-((2-((tert-butoxycarbonyl)amino)ethyl)carbamoyl)(3-(4-carboxyphenyl)-1,2,3-oxadiazol-3-ium-5-yl)amide

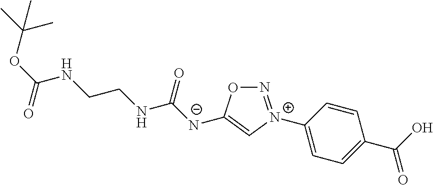 [SMILES: CC(C)(C)OC(=O)NCCNC(=O)[N-]c1c[n+](-c2ccc(C(=O)O)cc2)no1]

To a solution of ((2-((tert-butoxycarbonyl)amino)ethyl)carbamoyl)(3-(4-(ethoxycarbonyl)phenyl)-1,2,3-oxadiazol-3-ium-5-yl)amide (65 mg, 0.155 mmol) in EtOH (1 mL)/THF (1 mL) was added NaOH (5 eq). The resulting suspension was stirred at room temperature. After 2 h, the mixture was diluted with water then extracted with AcOEt (3 times). The aqueous phase was acidified with HCl (2 N) until pH ca. 2. Then it was extracted with AcOEt (3 times). The combined organic layers were dried (MgSO₄) and concentrated under reduced pressure to give the desired product.

¹H NMR (DMSO, 400 MHz, ppm): δ=9.35 (s, 1H), 8.10 (d, J=8.4 Hz, 2H), 7.99 (d, J=8.4 Hz, 2H), 6.94 (t, J=5.2 Hz, 1H);

¹³C NMR (DMSO, 101 MHz, ppm): δ=172.2, 167.7, 161.3, 156.0, 141.3, 135.2, 131.0 (2C), 121.9 (2C), 102.6, 78.0, 40.5, 39.2, 9.6 (3C);

MS (ESI) m/z: [M+H]+=392.3.

### Step (d): synthesis if J-((2-ammonioethyl)carbamoyl)(3-(4-carboxyphenyl)-1,2,3-oxadiazol-3-ium-5-yl)amide 2,2,2-trifluoroacetate

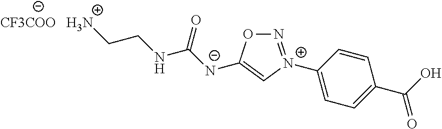 [SMILES: "CF3COO" ; [NH3+]CCNC(=O)[N-]c1c[n+](-c2ccc(C(=O)O)cc2)no1]

To a solution of ((2-((tert-butoxycarbonyl)amino)ethyl)carbamoyl)(3-(4-carboxyphenyl)-1,2,3-oxadiazol-3-ium-5-yl)amide (1 equiv.) in DCM (2 mL) was added TFA (0.5 mL). The resulting solution was stirred at room temperature. After 4 h, the solvent was evaporated under reduced pressure (with re-solubilisation in MeOH and re-evaporation 2 times in order to achieve full elimination of TFA).

¹H NMR (MeOD, 400 MHz, ppm): δ=9.21 (s, 1H), 8.37 (d, J=8.4 Hz, 2H), 8.20 (d, J=8.4 Hz, 2H), 3.20-3.10 (m, 4H);

MS (ESI) m/z: [M+H]⁺=292.1.

### Step (e): Synthesis of K=Im13

To a homogeneous solution of ((2-ammonioethyl)carbamoyl)(3-(4-carboxyphenyl)-1,2,3-oxadiazol-3-ium-5-yl)amide 2,2,2-trifluoroacetate (1 equiv.) in dry DMF (1 mL) was added TEA (3 equiv.). The mixture was cooled at 0° C. and then 2,5-dioxopyrrolidin-1-yl 3-(2,5-dioxo-2,5-dihydro-1H-pyrrol-1-yl)propanoate (1 equiv.) was added to the mixture. After 18 h, the mixture was concentrated under vacuum. The residue was dissolved in EtOAc (100 mL) and extracted with 0.1 N HCl (2×). The organic layer was washed with brine (2×) and dried (Na2SO4) and concentrated to give the desired product.

¹H NMR (MeOD, 400 MHz, ppm): δ=9.27 (s, 1H), 8.39 (d, J=8.6 Hz, 2H), 8.23 (d, J=5.6 Hz, 2H), 6.81 (s, 2H), 3.78 (t, J=6.7 Hz, 2H), 3.37-3.32 (m, 4H), 2.46 (t, J=6.7 Hz, 2H);

MS (ESI) m/z: [M+H]⁺=443.1.

### Im14: ((4-(cyanoethynyl)phenyl)carbamoyl)(3-(4-(ethoxycarbonyl)phenyl)-1,2,3-oxadiazol-3-ium-5-yl)amide

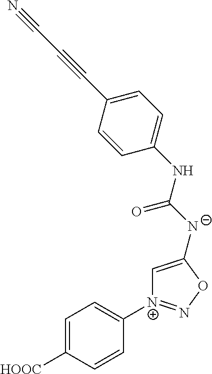 [SMILES: N#CC#Cc1ccc(NC(=O)[N-]c2c[n+](-c3ccc(C(=O)O)cc3)no2)cc1]

This compound was prepared according to a process comprising the following successive steps.

### Step 1: Synthesis of 2-((4-iodophenyl)amino)acetonitrile

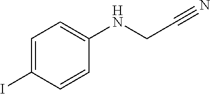 [SMILES: N#CCNc1ccc(I)cc1]

To a 250 mL round bottom flask was charged with 4-iodoaniline (5 g, 22.83 mmol, 1 equiv.), NaI (3.42 g, 22.83 mmol, 1 equiv.), K₂CO₃ (3.7 g, 27.39 mmol, 1.2 equiv.) and MeCN (60 mL) was added dropwise chloroacetonitrile (2.86 mL, 45.65 mmol, 2 equiv.) the mixture was stirred at reflux under nitrogen atmosphere.

After 48 h, the mixture was allowed to cool at room temperature and filtered through a fritted glass filter with AcOEt (200 mL). The solution was then washed with NaClₛₐₜ (100 mL). The combined organic layers were dried (MgSO₄) and concentrated under reduced pressure. The resulting material was purified though flash chromatography (100% cyclohexane to 80% cyclohex: 20% AcOEt) to afford 5.46 g (21.16 mmol, 92% yield) of analytically pure compound.

¹H NMR (CDCl₃, 400 MHz, ppm): δ=7.52 (d, J=8.8 Hz, 2H), 6.48 (d, J=8.8 Hz, 2H), 4.07 (s, 2H);

¹³C NMR (CDCl₃, 101 MHz, ppm): δ=144.6, 138.2 (2H), 116.5, 115.7 (2H), 81.5, 32.4;

MS (ESI) m/z: [M+H]⁺=259.0;

### Step 2: Synthesis of 5-amino-3-(4-iodophenyl)-1,2,3-oxadiazol-3-ium chloride

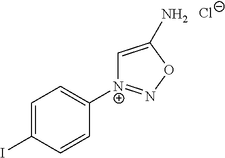 [SMILES: [Cl-] ; Nc1c[n+](-c2ccc(I)cc2)no1]

To a solution of 2-((4-iodophenyl)amino)acetonitrile (3.5 g, 13.56 mmol, 1 equiv.) in THF (20 mL) stirred at room temperature was added dropwise isopentyl nitrite (5.5 mL, 40.68 mmol, 3 equiv.). The mixture was stirred until full conversion to the corresponding nitroso aniline was achieved (TLC monitoring). Afterword, the solvent was evaporated to dryness under high vacuum to eliminate the excess of nitrite. Then the residue was dissolved in dry THF (10 mL) and a solution of dry HCl in dioxane 4 M (34 mL, 10 equiv.) was added. The mixture was stirred at room temperature. After 16 h, the precipitate formed was filtered and washed with Et₂O (150 mL) to afford analytically pure product.

¹H NMR (DMSO, 400 MHz, ppm): δ=10.10 (s, 2H), 8.69 (s, 1H), 8.14 (d, J=8.4 Hz, 2H), 7.84 (d, J=8.4 Hz, 2H);

¹³C NMR (DMSO, 101 MHz, ppm): δ=169.4, 139.1 (2C), 132.4, 124.4 (2C), 102.3, 101.5;

MS (ESI) m/z: [M-Cl]⁺=288.2.

### Step 3: Synthesis of (tert-butoxycarbonyl)(3-(4-iodophenyl)-1,2,3-oxadiazol-3-ium-5-yl)amide

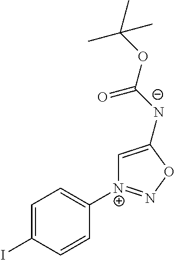 [SMILES: CC(C)(C)OC(=O)[N-]c1c[n+](-c2ccc(I)cc2)no1]

To a solution of 5-amino-3-(4-iodophenyl)-1,2,3-oxadiazol-3-ium chloride (4.08 mmol, 1 equiv.), NaHCO₃ (6.12 mmol, 1.5 equiv.) and DMAP (0.41 mmol, 10 mol %) and THF (30 mL) was added dropwise di-tert-butyl dicarbonate (6.12 mmol, 1.5 equiv.). The resulting mixture was stirred at room temperature for 48 h. The heterogeneous mixture was quenched with NH₄C1 (35 mL) and extracted with AcOEt (3*30 mL). The combined organic layers were dried over MgSO₄ and concentrated under reduced pressure. The crude material was purified though automated flash chromatography (from 100% cyclohexane to 80% cyclohexane/20% AcOEt) to afford 1.36 g (3.51 mmol, 86% yield) of analytically pure compound.

¹H NMR (CDCl₃, 400 MHz, ppm): δ=8.10 (s, 1H), 7.98, (d, J=8.7 Hz, 2H), 7.52 (d, J=8.7, 2H) 1.51 (s, 9H);

¹³C NMR (CDCl₃, 101 MHz, ppm): δ=174.3, 160.2, 139.7 (2C), 133.3, 122.7 (2C), 102.3, 99.48, 79.4, 28.1 (3C);

IR (v, cm⁻¹): 3170, 3092, 2973, 1783, 1752, 1660, 1596, 1580, 1488, 1364, 1291, 1048, 1005, 964, 826, 728;

MS (ESI) m/z: [M+H]=388.1;

Mp: 134-136° C.

### Step 4: Synthesis of (tert-butoxycarbonyl)(3-(4-(ethoxycarbonyl)phenyl)-1,2,3-oxadiazol-3-ium-5-yl)amide

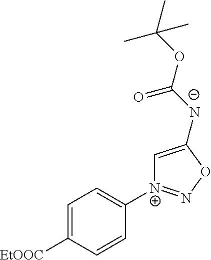 [SMILES: CCOC(=O)c1ccc(-[n+]2cc([N-]C(=O)OC(C)(C)C)on2)cc1]

(tert-butoxycarbonyl)(3-(4-iodophenyl)-1,2,3-oxadiazol-3-ium-5-yl)amide (300 mg, 0.774 mmol, 1 equiv.) was dissolved in a mixture of absolute EtOH (2.5 mL) and triethylamine (2.5 mL). [PdCl₂(PPh₃)₂] (81 mg, 0.116 mmol, 15 mol %) was added and the reaction mixture was kept under a CO atmosphere for 2.5 h and heated at 60° C.

The resulting suspension was cooled to room temperature and filtered through Celite eluting with ethyl acetate, and the inorganic salts were removed. The filtrate was concentrated and purification of the residue by silica gel column chromatography gave the desired product. The resulting residue was purified by chromatography (from 100% cyclohexane to 70% cyclohexane/30% AcOEt) to afford 183 mg (0.550 mmol, 71% yield) of analytically pure compound.

¹H NMR (CDCl₃, 400 MHz, ppm): δ=8.29 (d, J=9.0 Hz, 2H), 8.16 (s, 1H), 7.87 (d, J=9.0 Hz, 2H), 4.43 (q, J=7.0 Hz, 2H), 1.51 (s, 9H), 1.42 (t, J=7.0 Hz, 3H);

¹³C NMR (CDCl₃, 101 MHz, ppm): δ=174.6, 164.3, 160.6, 136.6, 134.7, 131.6 (2C), 121.4 (2C), 102.5, 79.2, 62.0, 28.1 (3C), 14.2;

IR (v, cm⁻¹): 3159, 2979, 1784, 1716, 1662, 1586, 1366, 1271, 1152, 1106, 1048, 1007, 963, 855, 769, 728, 689;

MS (ESI) m/z: [M+H]=334.3;

Mp: 151-153° C.

### Step 5: Synthesis of ((4-(cyanoethynyl)phenyl)carbamoyl)(3-(4-(ethoxycarbonyl)phenyl)-1,2,3-oxadiazol-3-ium-5-yl)amide

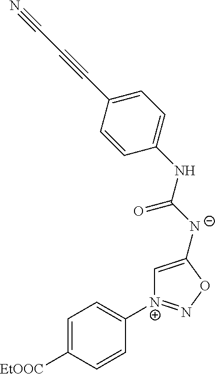 [SMILES: CCOC(=O)c1ccc(-[n+]2cc([N-]C(=O)Nc3ccc(C#CC#N)cc3)on2)cc1]

To a solution of (tert-butoxycarbonyl)(3-(4-(ethoxycarbonyl)phenyl)-1,2,3-oxadiazol-3-ium-5-yl)amide (91 mg, 0.273 mmol) in DCM (5 mL) was added TFA (0.5 mL). The mixture was stirred at room temperature for 3 hours. After it was reached full de-protection (monitoring by LCMS), the solvent was evaporated under reduced pressure (with re-solubilisation in MeOH and re-evaporation 2 times in order to achieve full elimination of TFA). In a separate flask, to a heterogeneous solution of triphosgene (0.33 equiv.) and 3-(4-aminophenyl)prop-2-ynenitrile (1 equiv.) in DCM (2 mL) was added, drop-wise at 0° C., an aqueous solution of NaHCO₃ (3 equiv. in 1.5 mL of water). The mixture was stirred at r.t. for 30 min, then the deprotected amine derivative was added in DCM. The mixture was stirred at room temperature. After 18 h, the yellow heterogeneous mixture was diluted in Et₂O (50 mL) and filtered. The yellow ppt was washed with Et₂O, collected and dried under vacuum, to afford the desired product in 44% yield.

¹H NMR (DMSO, 400 MHz, ppm): δ=9.98 (s, 1H), 8.72 (s, 1H), 8.23-8.22 (m, 4H), 7.77 (d, J=8.5 Hz, 2H), 7.67 (d, J=8.5 Hz, 2H), 4.37 (q, J=7.0 Hz, 2H), 1.34 (t, J=7.0, 3H);

IR (v, cm⁻¹): 3345, 3144, 2256, 1697, 1651, 1571, 1616, 1513, 1491, 1431, 1311, 1279, 1241, 1179, 1009, 931, 836, 535;

MS (ESI) m/z: [M+H]⁺=402.1.

### Step 6: Synthesis of Im14-(3-(4-carboxyphenyl)-1,2,3-oxadiazol-3-ium-5-yl)((4-(cyanoethynyl)phenyl)carbamoyl)amide

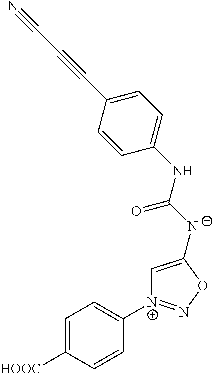 [SMILES: N#CC#Cc1ccc(NC(=O)[N-]c2c[n+](-c3ccc(C(=O)O)cc3)no2)cc1]

To a solution of ((4-(cyanoethynyl)phenyl)carbamoyl)(3-(4-(ethoxycarbonyl)phenyl)-1,2,3-oxadiazol-3-ium-5-yl)amide (55 mg, 0.15 mmol) in EtOH (1 mL)/THF (1 mL) was added NaOH (5 eq). The resulting suspension was stirred at room temperature. After 2 h, the mixture was diluted with water then extracted with AcOEt (3 times). The aqueous phase was acidified with HCl (2 N) until pH ca. 2. Then it was extracted with AcOEt (3 times). The combined organic layers were dried (MgSO₄) and concentrated under reduced pressure to give the desired product in 7% yield.

¹H NMR (DMSO, 400 MHz, ppm): δ=9.98 (s, 1H), 8.72 (s, 1H), 8.13-8.02 (m, 4H), 7.77 (d, J=8.5 Hz, 2H), 7.67 (d, J=8.5 Hz, 2H).

MS (ESI) m/z: [M+H]⁺=374.2.

### Example 2: Kinetic Study of the Reaction of Iminosydnones with Cyclic Alkynes

### Experimental

Reactions of iminosydnones Im 2 and Im 5 as described in Table 1 below with tetramethylthiacycloheptyne (TMTH) was carried out in PBS buffer (0.1M, pH 7.4) at 100 μM concentration of iminosydnone and 150 μM concentration of cyclooctyne TMTH using the following procedure:

To 900 μL of PBS buffer were added 10 μL of the solution of benzamide (internal standard, 100 mM in DMSO), 1 μL of the solution of iminosydnone (100 mM in DMSO) and 1.5 μL of the solution of TMTH (100 mM in DMSO). The reaction mixture was injected in HPLC and the conversion was followed by measuring the normalized iminosydnone peak area.

### Results:

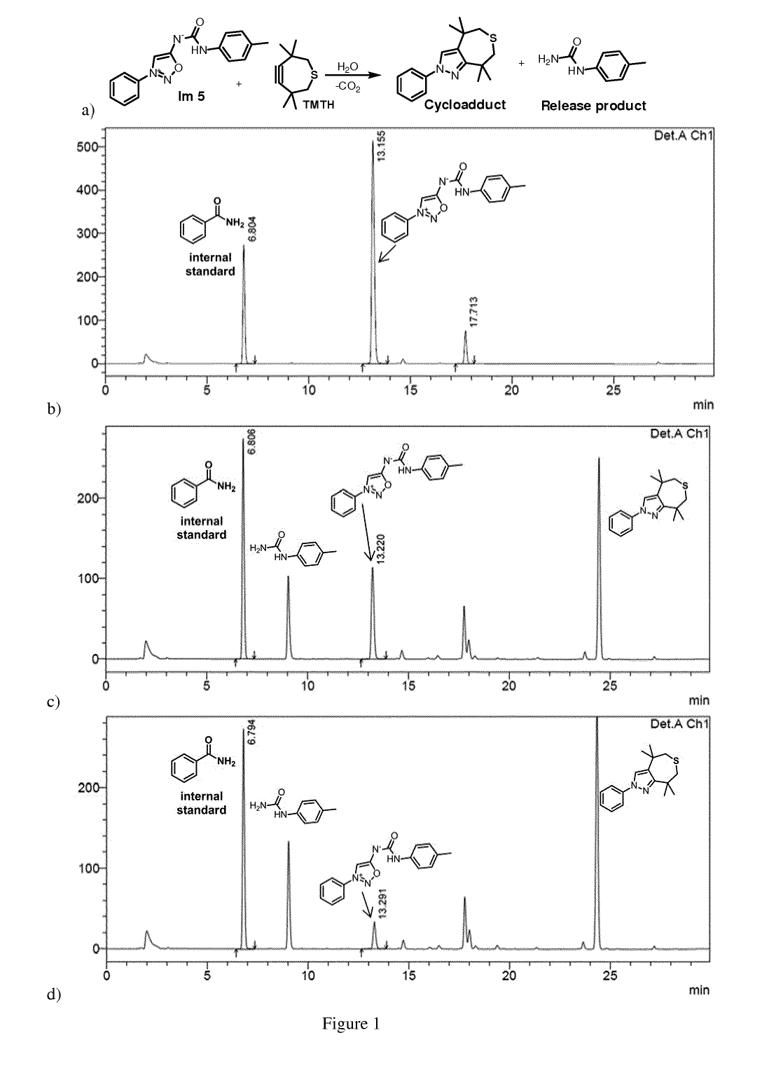

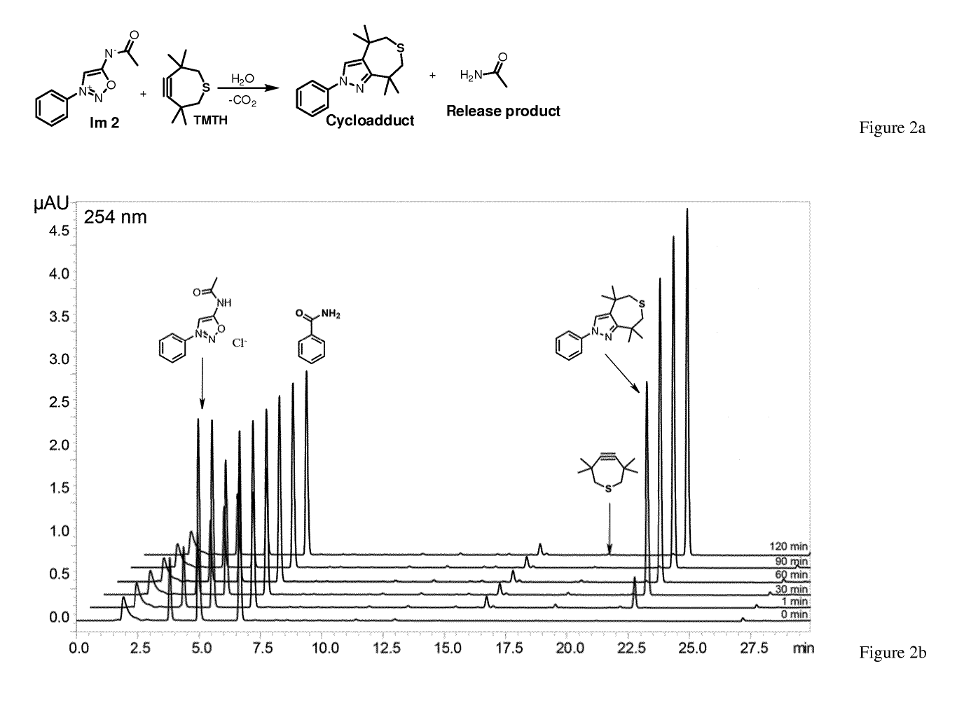

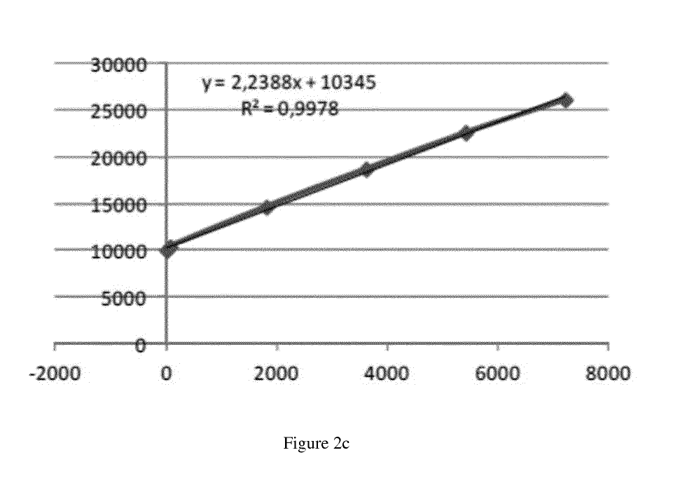

FIGS. 1 and 2 present the HPLC monitoring of the reaction between the cyclic alkyne TMTH and iminosydnones Im5 and Im 2, respectively. The reaction has been conducted at 100 μM in PBS buffer (pH 7.4). The results indicated an almost complete reaction after 30 min proving that the reaction is fast enough to be useful for bioconjugation and release applications. The presence of a F group on the phenyl group would clearly not detrimentally affect these results. One can expect similar or even improved kinetics for the iminosydnones according to the invention.

### Example 3: Kinetic Study of Different Iminosydnones

The kinetics of the coupling reaction of different iminosydnones with two cyclic alkynes was studied, according to the following scheme.

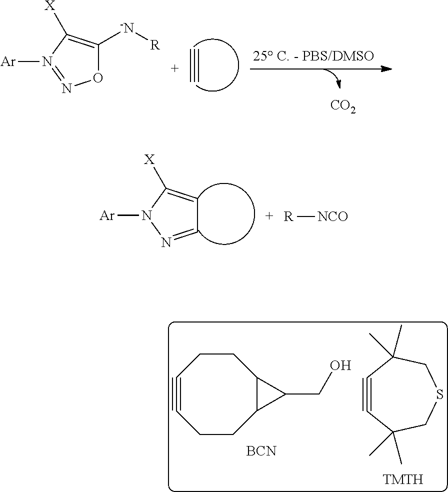

### Experimental

Reactions of iminosydnones with BCN (Biclyclononyne) or TMTH were carried out in PBS/DMSO (9:1) mixtures at 100 μM concentration of sydnones and 150 μM concentration of BCN or TMTH using the following procedure:

To 900 μL of phosphate buffered saline (PBS, 100 mM) was added 87.5 μL of DMSO, 10 μL of the solution of benzamide (internal standard, 100 mM in DMSO), 1 μL of the solution of iminosydnone (100 mM in DMSO) and 1.5 μL of the solution of BCN or TMTH (100 mM in DMSO). The reaction mixture was injected in HPLC every 30 min and the conversion was followed by measuring the normalized sydnone peak area.

### Results:

Table 1 below presents the kinetic constant values K for these reactions, depending on the Ar group, the R group and the X group. Only iminosydnones 7, 13 and 14 are according to the invention.

It can be seen that Im7, which is according to this invention, provides a higher kinetic constant value, both with BCN and with TMTH, than similar iminosydnones which bear either no F group (see Im5) or a different F group (see Im6). These results further show that the iminosydnones comprising a carbamate function at F′ position (compare Im 11 with Im3) and the iminosydnones comprising a halogen atom in position 4 (compare Im9 with Im10 and Im11 with Im12) afford the fastest reactions. In particular, 4-bromo-6-carbamate iminosydnones were found to react with the cyclooctyne BCN with a constant of 0.15 M⁻¹·sec⁻¹ which allows the reaction to proceed at concentration as low as 1 μM.

Second order reaction rate was determined by plotting ln([A]/[B])/([A]−[B]) versus time and analyzing by linear regression (Equation). Second order rate constant corresponds to the determined slope.

$$\frac{ln ( \frac{[ A ]}{[ B ]} )}{[ A ] - [ B ]} = kt + const \\ [ A ] \text{-} concentration of iminosydnones ( M ) ; \\ [ B ] \text{-} concentration of BCN ( M ) ; \\ t \text{-} reaction time ( sec ) ; \\ k \text{-} reaction rate ( {M}^{- 1} \cdot {sec}^{- 1} ) \qquad Equation$$

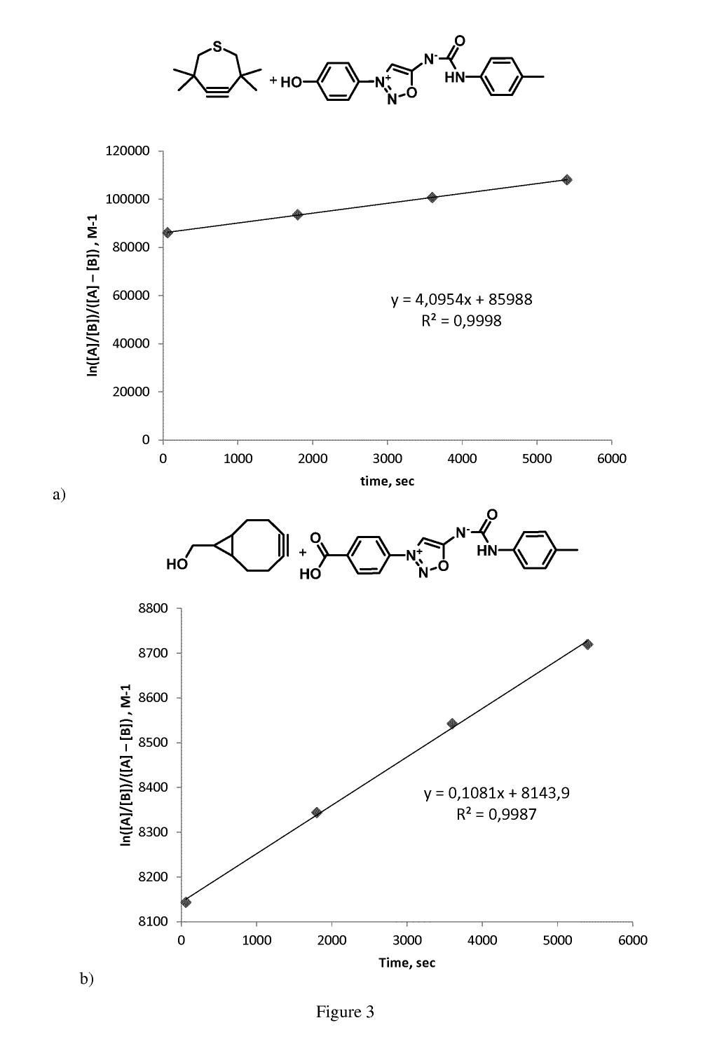

Linear regression curves for sydnones Im2, Im6 and Im7 are illustrated in FIGS. 2 and 3.

1-14. (canceled)

15. A process for the preparation of a functionalized compound of interest C1 of formula (II): 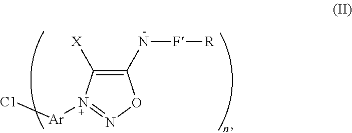
wherein n is an integer from 1 to 100, comprising the step of contacting a compound of interest C1 selected from an antibody, a protein, a drug, a fluorophore, a group of atoms comprising at least one radioactive atom, a DNA fragment, a nanoparticle and a polymer, with an iminosydnone of formula (I): 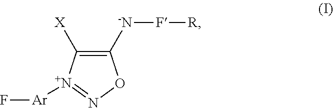
wherein:
C1 bears a reactive group which is able to react with F,
X is selected from the group consisting of a hydrogen atom, a halogen atom, an aryl diazo group, an alkyl group, an aryl group, an alkenyl group, an alkynyl group, an alkoxy group, a thioether group and an amino group,
F is selected from the group consisting of:
a carboxylic acid COOH group,
a thiol SH group,
a maleimide 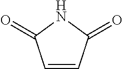
group,
an activated ester,
a halogen atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups,
a hydroxylamine (—ONH₂) group,
a hydrazine (—NH—NH₂) group,
an azido (—N₃) group,
a diazonium (—N₂₊) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′″ group, wherein R′″ is an alkyl group, and
an alkyl group substituted by at least one of said groups,
F′ is a carbonyl group (C═O), a sulfonyl group (SO₂) or a phosphoryl group (P═O),
R is selected from an optionally substituted aryl group, an optionally substituted alkyl, alkenyl or alkynyl group, an optionally substituted alkoxy group, an optionally substituted thioether group, an optionally substituted amino group, wherein the alkyl, alkenyl and/or alkynyl groups may be interrupted by at least one heteroatom selected from nitrogen, oxygen and sulphur atoms, and wherein said substituents are one or more groups selected from:
a carboxylic acid COOH group,
a thiol SH group,
a maleimide 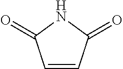
group,
an activated ester,
a halogen atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups,
a hydroxylamine (—ONH₂) group,
a hydrazine (—NH—NH₂) group,
an azido (—N₃) group,
a diazonium (—N₂₊) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′″ group, wherein R′″ is an alkyl group, or
a linker bearing at least one of the above substituents, and
Ar is an optionally substituted aromatic group.

16. Process according to claim 15, wherein F is selected from the group consisting of a carboxylic acid COOH group, an activated ester and an alkyl group substituted by at least one of these groups.

17. Process according to claim 15, comprising a preliminary step of covalent bonding of the reactive group to C1.

18. Process according to claim 15, wherein the reactive group is selected from the group consisting of:
a carboxylic acid COOH group,
a thiol SH group,
a maleimide 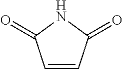
group,
an activated ester,
a halogen atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups,
a hydroxylamine (—ONH₂) group,
a hydrazine (—NH—NH₂) group,
an azido (—N₃) group,
a diazonium (—N₂₊) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′″ group, wherein R′″ is an alkyl group.

19. Process according to claim 18, wherein the reactive group is an amino group or a thiol group.

20. Functionalized compound of interest C1 of formula (II): 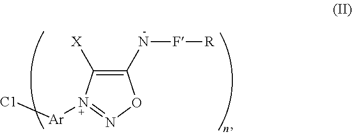
wherein n, Ar, X, F′, C1 and R are as defined in claim 15 and wherein C1 and Ar are covalently linked by a functional group.

21. Conjugate of formula (IV): 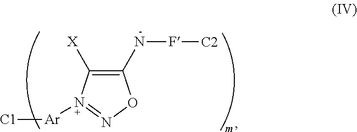
wherein
m is an integer from 1 to 100,
C2 is a compound of interest selected from the group consisting of a fluorophore, a group of atoms comprising at least one radioactive atom, a drug, an antibody, a protein, a DNA fragment, a nanoparticle and a polymer,
C1, X, Ar and F′ are as defined in claim 15,
C1 and Ar are covalently linked by a functional group, and
C2 and F′ are covalently linked by a functional group.

22. Process for the preparation of a conjugate according to claim 21, comprising the step of contacting a compound of formula (II) with the compound of interest C2, wherein C2 bears a reactive group which is able to react with R.

23. Process for the preparation of a conjugate of formula (V): 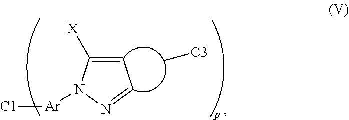
wherein p is an integer from 1 to 100, C1, Ar and X are as defined in claim 15 and C3 is a third compound of interest,
comprising a step of contacting a compound of formula (VI): 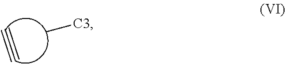
wherein C3 is a third compound of interest selected from the group consisting of a fluorophore, a group of atoms comprising at least one radioactive atom, a drug, an antibody, a protein, a DNA fragment, a nanoparticle and a polymer, with a compound of formula (II) or with a conjugate of formula (IV).

24. Process of preparation according to claim 23, wherein the process comprises a preliminary step of covalent bonding of a strained alkyne moiety to the compound of interest C3.

25. Conjugate of formula (V): 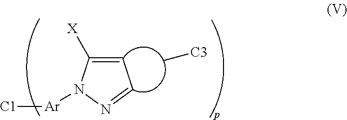
wherein p is an integer from 1 to 100, C1, C3, Ar and X are as defined in claim 23.

26. Process for releasing a derivative of a compound of interest C2 of formula (VII): 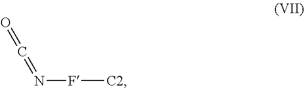
comprising a step of contacting a conjugate of formula (IV) according to claim 21 with a compound comprising a strained alkyne moiety or a compound of formula (VI): 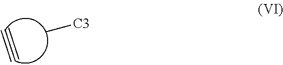
wherein C3 is a third compound of interest selected from the group consisting of a fluorophore, a group of atoms comprising at least one radioactive atom, a drug, an antibody, a protein, a DNA fragment, a nanoparticle and a polymer.

27. Iminosydnone of formula (I′): 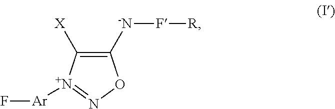
wherein
X is a halogen atom,
F is selected from the group consisting of:
a carboxylic acid COOH group,
a thiol SH group,
a maleimide 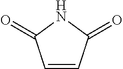
group,
an activated ester,
a halogen atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups,
a hydroxylamine (—ONH₂) group,
a hydrazine (—NH—NH₂) group,
an azido (—N₃) group,
a diazonium (—N₂⁺) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′″ group, wherein R′″ is an alkyl group, and
an alkyl group substituted by at least one of said groups,
F′ is a carbonyl group (C═O), a sulfonyl group (SO₂) or a phosphoryl group (P═O),
R is selected from an optionally substituted aryl group, an optionally substituted alkyl, alkenyl or alkynyl group, an optionally substituted alkoxy or aralkyloxy group, an optionally substituted thioether group, an optionally substituted amino group, wherein the alkyl, alkenyl and/or alkynyl groups may be interrupted by at least one heteroatom selected from nitrogen, oxygen and sulphur atoms and wherein said substituents are one or more groups selected from:
a carboxylic acid COOH group,
a thiol SH group,
a maleimide 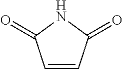
group,
an activated ester,
a halogen atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups,
a hydroxylamine (—ONH₂) group,
a hydrazine (—NH—NH₂) group,
an azido (—N₃) group,
a diazonium (—N₂+) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′″ group, wherein R′″ is an alkyl group, or
a linker bearing at least one of the above substituents, and
Ar is an optionally substituted aromatic group.

28. Iminosydnone according to claim 27, wherein F′ is a C═O group and R is an amino group, forming a urea moiety with the F′ group, and X is a halogen atom selected from chlorine and bromine.
